(19) United States
(12) Patent Application Publication
(10) Pub. No.: US 2006/0129246 A1
(43) Pub. Date: Jun. 15, 2006

(54) Mobile bearing unicondylar knee prosthesis

(75) Inventor: Scott J. Steffensmeier, Warsaw, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/351,795

(22) Filed: Feb. 10, 2006

Related U.S. Application Data
(63) Continuation of application No. 10/969,514, filed on Oct. 20, 2004.

(51) Int. Cl. A61F 2/38

(52) U.S. Cl. 623/20.29; 623/20.3

(57) ABSTRACT

A mobile bearing unicondylar tibial knee prosthesis for repairing a damaged knee joint is provided. The prosthesis includes a tibial base plate and a tibial meniscal component supported on the tibial base plate in relative sliding relationship. The relative sliding becomes more constrained as the tibial meniscal component moves toward the sides of the base plate.

### FIELD OF THE INVENTION

The invention relates to prostheses for the knee joint. In particular, the invention relates to unicondylar mobile bearing knee joint prostheses.

### BACKGROUND

Degenerative and traumatic damage to the articular cartilage of the knee joint can result in pain and restricted motion. Prosthetic joint replacement is frequently utilized to alleviate the pain and restore joint function. In a total knee replacement, all of the articulating compartments of the joint are repaired with prosthetic components. However, often only one compartment of the knee joint, typically the medial compartment, is impaired. Thus, in a unicondylar knee replacement, only the damaged compartment is repaired with prosthetic bearing components. Unicondylar knee replacement can be less invasive and can have a shorter recovery time than total knee replacement. In repairing the damaged portions of the knee, it is desirable to restore the joint as nearly as possible to normal anatomic functioning.

### SUMMARY

The present invention provides a mobile bearing unicondylar knee prosthesis for repairing a damaged knee joint including a tibial base plate and a tibial meniscal component.

In one aspect of the invention a tibial base plate includes an inferior surface for engaging a tibia and a superior surface. The superior surface has a relatively flat central portion and relatively more sloping anterior and posterior portions sloping upwardly away from the central portion. The tibial meniscal component includes an inferior surface slidingly engageable with the superior surface of the base plate. The tibial base plate supports the tibial meniscal component in unconstrained sliding relationship over the central portion of the base plate and with increasing constraint over the sloping anterior and posterior portions.

In another aspect of the invention, the tibial base plate has an inferior surface for engaging a tibia and a superior surface. The superior surface includes a central portion defining a concavity. The tibial meniscal component includes an inferior surface having a convex portion engageable with the concavity of the base plate in pivoting and sliding articular relationship. The meniscal component further has at least one pivot limiting portion extending outwardly away from the convex portion and overhanging the superior surface of the tibial base plate.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side elevation view of a prosthesis according to the present invention including a tibial base plate, a tibial bearing component, and a femoral component;

FIG. 2 is a front elevation view of the prosthesis of FIG. 1;

FIG. 3 is a side elevation view of the prosthesis of FIG. 1 depicting an alternate engagement between the tibial base plate and tibial bearing component;

FIG. 4 is a front elevation view of the prosthesis of FIG. 3;

FIG. 5 is a side elevation view of the prosthesis of FIG. 1 depicting an alternate engagement between the tibial base plate and tibial bearing component; and

FIG. 6 is a side elevation view of the prosthesis of FIG. 5 showing the tibial bearing component displaced to one side.

### DESCRIPTION OF THE ILLUSTRATIVE EXAMPLES

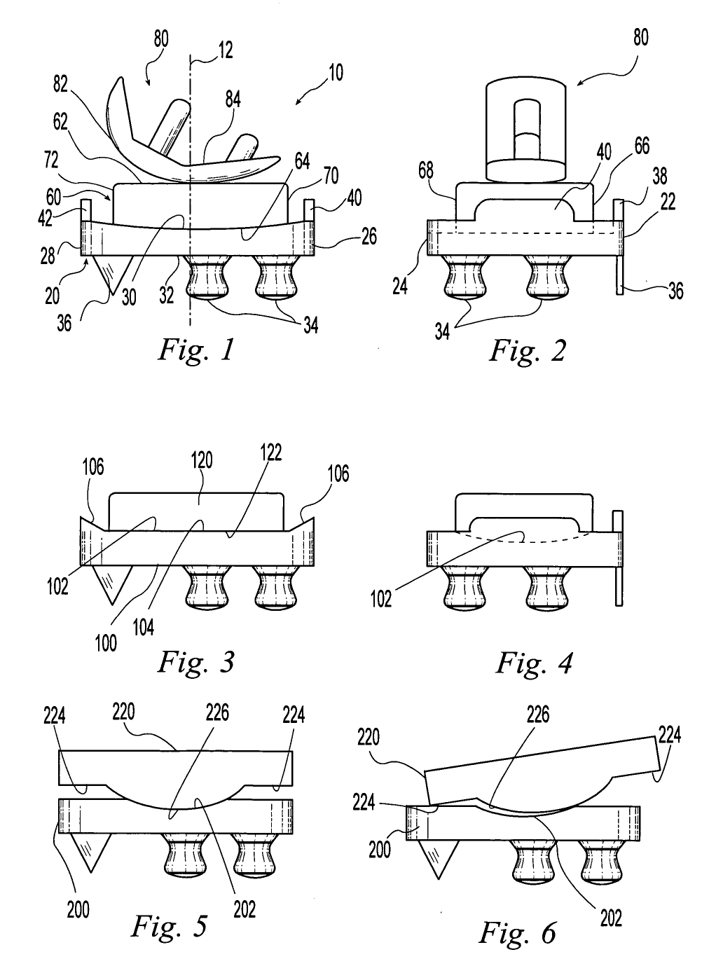

The mobile bearing unicondylar knee prosthesis of the present invention will be explained with reference to FIGS. 1-6. The drawings depict a mobile bearing unicondylar knee prosthesis for replacing one side of a knee joint.

FIGS. 1-2 depict an illustrative mobile bearing unicondylar knee prosthesis 10 including a tibial base plate component 20, a tibial meniscal component 60, and a femoral component 80. The base plate 20 has inner and outer sides 22, 24 corresponding to the medial-lateral plane, anterior and posterior sides 26, 28 corresponding to the anterior-posterior plane, a superior surface 30 and an inferior surface 32. The base plate 20 includes a smooth superior surface 30 for sliding engagement with the meniscal component 60 and an inferior surface 32 for engaging the bone. The superior surface 30 is upwardly concave in at least one plane. In this context, concave is used to denote a superior surface 30 in which a portion of the periphery of the surface 30, i.e. nearer to the inner, outer, anterior, or posterior sides 22, 24, 26, 28 rises further superiorly, or upwardly in the direction of the vertical axis 12, than a portion of the surface nearer the middle of the surface. The illustrative tibial base plate 20 of FIGS. 1-2 is concave along a curve rising both anteriorly and posteriorly in the anterior-posterior plane and is flat in the medial-lateral plane. Alternatively, the base plate 20 may be concave in the medial-lateral plane or in both planes. The inferior surface 32 may optionally include positive bone fixation features such as pegs 34 and/or a fin 36 for insertion into the bone of the proximal tibial for enhanced fixation.

The meniscal component 60 includes a superior surface 62 for articulation with the femoral component 80, an inferior surface 64 for sliding engagement with the superior surface 30 of the base plate 20, an inner side 66, an outer side 68, an anterior side 70, and a posterior side 72. The inferior surface 64 of the meniscal component 60 may be flat or curved and it may have a curve different from the superior surface 30 of the tibial plate 20 or it may have a conforming complimentary curve as shown in the illustrative example of FIG. 1. The meniscal component 60 rests on the base plate 20 and is generally free to slide relative to the base plate 20.

The femoral component 80 includes an inferior surface 82 for articulation with the superior surface 62 of the meniscal component 60 and a superior surface 84 for engaging bone. The femoral component 80 rests on the meniscal component 60 and is generally free to slide relative to the meniscal component 60.

In use the components are installed in a patient's knee between a tibia and a femur and are oriented as shown in FIGS. 1 and 2. The capsular ligaments and the patient's body weight tend to press the components together into engagement along the vertical axis 12. As the patient flexes the knee joint, the components 20, 60, 80 will slide and rotate relative to one another. As the meniscal component 60 slides on the concave superior surface 30 of the tibial base plate 20 it will be displaced superiorly, or upwardly along the vertical axis 12, when it moves over any portion that rises superiorly. This upward displacement of the meniscal component 60 will in turn move the femoral component superiorly such that the tibia and femur are moved apart. Because the patient's body weight and ligament tension resist displacement of the tibia and femur away from one another, the meniscal component will encounter increasing resistance to sliding along rising portions. Thus, the concavity of the superior surface 30 of the tibial base plate 20 acts as a constraint on the motion of the meniscal component 60 relative to the base plate 20. Portions of the superior surface 30 that rise further from the center of the superior surface 30 will provide more constraint than portions the rise a smaller distance. The concavity of the superior surface 30 may be varied from zero, or flat, to a large value, or steeply sloped, to provide the desired amount of constraint. The concavity may be symmetric in one or more planes or it may be asymmetric with more constraint being provided in one direction than another. In the illustrative tibial base plate 20 of FIGS. 1 and 2, the constraint is symmetric and increasing anteriorly and posteriorly to define a smooth curve as shown in FIG. 1 and symmetric and flat (no increase in constraint) in the medial-lateral plane as shown in FIG. 2.

The tibial base plate 20 may further include hard stops to more definitely limit the travel of the meniscal component 60 relative to the base plate 20. For example, an inside stop 38 adjacent the inner side 22 of the base plate 20 and/or an anterior stop 40 adjacent to the anterior side 26 and/or a posterior stop 42 adjacent to the posterior side 28 may be provided as a failsafe to the “soft” constraint of the concave superior surface 30.

FIGS. 3 and 4 show an alternative arrangement in which a tibial base plate 100 includes a concave superior surface 102 having a relatively flat middle portion 104 and relatively more sloping end portions 106. The middle portion 104 may be flat or it may just be relatively more flat than the end portions 106 which are relatively aggressively sloped. The end portions 106 may be linearly sloped or may be curved. In the illustrative arrangement of FIG. 3, the middle portion 104 of the superior surface 102 is flat and the end portions 106 are linear ramps. The meniscal component 120 has a flat inferior surface 122. In this arrangement, the meniscal component is relatively unconstrained in the center of its travel in the anterior-posterior plane with a rapid increase in constraint as it engages the end portions 106. As can be seen in FIG. 4, the superior surface is concavely dished to provide additional constraint to medial-lateral movement of the meniscal component 120.

FIGS. 5 and 6 show an alternative arrangement in which a tibial base plate 200 includes a concave superior surface 202 in which the concavity is located toward the center of the surface 202 such that even slight movement of the meniscal component 220 away from the center of the tibial base plate 200 meets increasing constraint in the anterior-posterior direction. The meniscal component 220 further includes at least one tilt limiting portion 224 that extends outwardly from a convex inferior surface 226 and overhangs the superior surface 202 to limit how much the meniscal component 220 may tilt relative to the tibial base plate 200. As the meniscal component 220 is displaced away from the center of the superior surface 202 and engages the concavity of the superior surface 202, it may begin to tilt as shown in FIG. 6. The tilt limiting portion 224 will engage the superior surface 202 to limit the amount of tilt of the meniscal component.

It will be understood by those skilled in the art that the foregoing has described illustrative embodiments of the present invention and that variations may be made to these embodiments without departing from the spirit and scope of the invention defined by the appended claims.

What is claimed is:

1. A unicondylar knee prosthesis comprising:
a tibial base plate having an inferior surface for engaging a tibia and a superior surface, the superior surface having a relatively flat central portion and relatively more sloping anterior and posterior portions sloping upwardly away from the central portion; and
a tibial meniscal component, the meniscal component including an inferior surface slidingly engageable with the superior surface of the base plate, the meniscal component further having a superior surface, the tibial base plate supporting the tibial meniscal component in unconstrained sliding relationship over the central portion of the base plate and with increasing constraint over the sloping anterior and posterior portions.

2. The prosthesis of claim 1 further comprising a femoral component, the femoral component having a superior surface for engaging a femur and an inferior surface engageable in articulating relationship with the superior surface of the meniscal component.

3. The prosthesis of claim 1 wherein the tibial base plate includes an inner side and an outer side aligned in a medial-lateral plane, and an anterior side and a posterior side aligned in an anterior-posterior plane, the superior surface of the base plate further being concave in the medial-lateral plane.

4. The prosthesis of claim 1 wherein the central portion of the superior surface of the tibial base plate comprises a flat surface.

5. The prosthesis of claim 4 wherein the anterior and posterior portions of the superior surface of the tibial base plate comprise linear ramps extending upwardly and outwardly from the central portion.

6. The prosthesis of claim 4 wherein the inferior surface of the tibial meniscal component comprises a flat surface conforming to the flat surface central portion of the tibial base plate.

7. The prosthesis of claim 1 wherein the tibial base plate further comprises inner, outer, anterior, and posterior sides and at least one hard stop adjacent to at least one of the inner, outer, anterior, and posterior sides, the hard stop extending upwardly from the superior surface of the tibial base plate, the hard stop comprising an inwardly directed face against which the tibial meniscal component abuts to positively limit the motion of the meniscal component relative to the tibial base plate.

8. The prosthesis of claim 7 wherein the tibial base plate comprises a hard stop adjacent each of the anterior, posterior, and inner sides.

9. A unicondylar knee prosthesis comprising:
a tibial base plate having an inferior surface for engaging a tibia and a superior surface, the superior surface comprising a central portion defining a concavity; and
a tibial meniscal component, the meniscal component including an inferior surface having a convex portion engageable with the concavity of the base plate in pivoting and sliding articular relationship, the meniscal component further comprising at least one pivot limiting portion extending outwardly away from the convex portion and overhanging the superior surface of the tibial base plate.

10. The prosthesis of claim 9 further comprising a femoral component, the femoral component having a superior surface for engaging a femur and an inferior surface in articulating engagement with the superior surface of the tibial articular surface.

11. The prosthesis of claim 9 wherein the articular relationship of the meniscal component and the tibial base plate is defined by discrete phases during knee joint articulation, the meniscal component initially pivoting within the concavity of the tibial base plate until the tilt limiting portion contacts the superior surface of the tibial base plate causing the meniscal component to transition to sliding articulation with the tibial base plate, the sliding articulation being progressively more constrained as the meniscal component moves away from the center of the concavity.
